(19) United States
(12) Patent Application Publication
(10) Pub. No.: US 2005/0175574 A1
(43) Pub. Date: Aug. 11, 2005

(54) Detergent cosmetic compositions comprising a surfactant, a drawing polymer and particles, and use thereof

(76) Inventors: Henri Samain, Bievres (FR); Geraldine Fack, Levallois (FR); Franck Giroud, Clichy (FR); Laurence Paul, Saint Leu La Foret (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/030,133

(22) Filed: Jan. 7, 2005

Related U.S. Application Data
(60) Provisional application No. 60/537,543, filed on Jan. 21, 2004.
(60) Provisional application No. 60/537,901, filed on Jan. 22, 2004.

(30) Foreign Application Priority Data
Jan. 7, 2004 (FR) 0450027
Jan. 7, 2004 (FR) 0450033

(51) Int. Cl. A61K 7/06; A61K 7/11; A61K 7/075; A61K 7/08

(52) U.S. Cl. 424/70.17; 424/70.22

(57) ABSTRACT

The present disclosure relates to novel washing compositions, such as hair compositions, comprising, in a cosmetically acceptable medium, at least one particle with a melting point of greater than 70° C., at least one detergent surfactant chosen from anionic, nonionic and amphoteric surfactants, and at least one drawing polymer, as well as a method of treatment using the compositions. These compositions have an improved styling effect.

This application claims benefit of U.S. Provisional Application No. 60/537,543, filed Jan. 21, 2004, and U.S. Provisional Application No. 60/537,901, filed Jan. 22, 2004.

The present disclosure relates to cosmetic compositions with improved properties, intended for cleaning, conditioning and/or styling keratin materials, such as the hair. In one embodiment, the composition comprises, in a cosmetically acceptable support, a washing base comprising at least one surfactant with detergent power, in which particles with a melting point of greater than 70° C. are also present in combination with drawing polymers. The present disclosure also relates to the use of the compositions in the abovementioned cosmetic application.

It is common practice to use detergent hair compositions (or shampoos) based essentially on standard surfactants of anionic, nonionic and/or amphoteric type, such as surfactants of anionic type, to clean and/or wash the hair. These compositions are applied to wet hair and the lather generated by massaging or rubbing with the hands removes, after rinsing with water, the various types of soiling initially present on the hair.

These base compositions may have good washing power, but the intrinsic cosmetic properties associated therewith may nevertheless remain fairly poor, owing, for example, to the fact that the relatively aggressive nature of such a cleaning treatment can, in the long run, lead to more or less pronounced damage to the hair fiber, this damage being associated, for example, with the gradual removal of the lipids or proteins contained in or on the surface of the fiber.

Thus, in order to improve the cosmetic properties of the above detergent compositions, such as those that are intended to be applied to sensitized hair (i.e., hair that has been damaged or made brittle, for example, due to the chemical action of atmospheric agents and/or hair treatments such as permanent-waving, dyeing or bleaching), it is now common practice to introduce additional cosmetic agents known as conditioners into these compositions. These conditioners are generally intended to repair or limit the harmful or undesirable effects induced by the various treatments or aggressions to which the hair fibers are subjected more or less repeatedly. These conditioners may, of course, also improve the cosmetic behavior of natural hair.

The conditioners most commonly used to date in shampoos are cationic polymers, which give washed, dry or wet hair an ease of disentangling, softness and smoothness that may markedly be better than those that may be obtained with corresponding cleaning compositions not containing such conditioners.

Moreover, it has for some time been sought to obtain conditioning shampoos that are capable of giving washed hair not only the cosmetic properties mentioned above but also, to a greater or lesser extent, styling, volume, shaping and hold properties. These washing shampoos with improved general cosmetic properties are often referred to for simplicity as “styling shampoos”, and this term is used in the description hereinbelow.

However, despite the progress made recently in the field of styling shampoos, these shampoos are not really completely satisfactory, and as such there is currently still a strong need for products that can give better performance with respect to one or more of the cosmetic properties mentioned above. For example, it is usually necessary to use a styling product after shampooing, to give the hair a shape and to fix the style.

The present disclosure is directed towards satisfying such a need.

Thus, after considerable research conducted in this matter, the present inventors have now found, entirely surprisingly and unexpectedly, that by combining water-insoluble particles with a melting point of greater than 70° C., as defined below, with certain polymers that have a particular drawing power in detergent compositions, it is possible to substantially and significantly improve the styling and hold properties, while at the same time maintaining their good intrinsic washing power and their cosmetic properties.

For example, these compositions make it possible to obtain very good hold and a certain amount of volume for the hair, for example, a styling effect similar to that obtained with a fixing styling gel used after shampooing. It is moreover found that the keratin fibers are strengthened.

Thus, disclosed herein are novel compositions for washing keratin materials such as the hair, comprising, in a cosmetically acceptable aqueous medium, at least one insoluble particle with a melting point of greater than 70° C., at least one detergent surfactant chosen from anionic, nonionic and amphoteric detergent surfactants and at least one polymer with a drawing power of greater than 5 cm.

Further disclosed herein are methods of cleaning, conditioning, caring for and/or styling keratin materials, such as the hair and the eyelashes, using the disclosed compositions.

Also disclosed herein are cosmetic processes for treating keratin materials, such as keratin fibers, and further such as the hair and the eyelashes, using the composition disclosed herein.

Even further disclosed herein is a shampoo comprising a composition according to the present disclosure.

However, other characteristics, aspects and advantages of the disclosure will emerge even more clearly on reading the description that follows, and also the concrete, but in no way limiting, examples intended to illustrate it.

As used herein, the “drawing power” of a polymer corresponds to the length of the polymer yarn obtained at the breaking point of the yarn according to the procedure defined below.

The drawing power of the polymers that may be used herein is the power measured for a composition containing (% by weight):

| | |
|---|---|
| Sodium lauryl ether sulfate oxyethylenated | 12.5% |
| with 2.2 mol of ethylene oxide | |
| Cocoamidopropylbetaine | 2.5% |
| Test polymer | 1% |
| Water qs | 100% |

The drawing power is measured using a TA-TX2 texture analyzer (Rheo/stable Micro Systems).

The measurement is performed after compression of the product:
Displacement of the disc (35 mm aluminium cylinder) at a speed of 2.5 mm/s and detection of the compression strength,
penetration into the product at the same speed to a depth of 10 mm,
removal of the probe at a speed of 2.5 mm/s, and
measurement of the displacement of the probe and detection of the breaking point of the product yarn.

As used herein, the polymers with a drawing power of greater than 5 cm are referred to as drawing polymers.

The polymers with a drawing power of greater than 5 cm are chosen, for example, from (a1) a dispersion of particles of a water-soluble polymer with a weight-average molecular mass of greater than 10⁶ in a saline aqueous solution, obtained by heterogeneous free-radical polymerization of water-soluble monomers with precipitation of the polymer formed, wherein at least one of the monomers is cationic, and (a2) an aqueous solution of a water-soluble polymer with a weight-average molecular mass of greater than 10⁶, obtained by heterogeneous free-radical polymerization of water-soluble monomers with precipitation of the polymer formed, wherein at least one of the monomers is cationic.

The high molecular weight water-soluble polymer is a cationic or amphoteric polyelectrolyte, i.e., a polyelectrolyte polymerized using at least one cationic monomer of formula (I) as described below.

In one embodiment, the water-soluble polymers are cationic. The term “cationic polymer” includes polymers comprising cationic monomers and possibly nonionic monomers.

As indicated above, the synthesis of the high molecular weight water-soluble polymers, used herein, takes place by heterogeneous free-radical polymerization of water-soluble monomers comprising at least one ethylenic unsaturation. The polymerization takes place in an aqueous solution of a mineral electrolyte (salt) having an ionic strength that is sufficient to cause precipitation of the polymer formed as soon as it has reached a certain molecular mass. This polymerization technique thus allows, by virtue of the well-known phenomenon of salting out, the preparation of saline aqueous dispersions of water-soluble polymer particles. The polymers thus synthesized are distinguished by a high weight-average molecular mass, which is generally greater than 10⁶.

The technique of heterogeneous free-radical polymerization in aqueous medium with precipitation of the polymer formed is described, for example, in U.S. Pat. No. 4,929,655, patent application EP 0 943 628 and International patent application WO 02/34796.

To ensure the stability of the dispersions of polymer particles during the synthesis and during storage, the polymerization is generally performed in the presence of a dispersant. This dispersant is, for example, a polyelectrolyte, which, unlike the high molecular weight polymer used herein, is soluble in the aqueous polymerization medium of high ionic strength.

This dispersing polyelectrolyte has, for example, a charge identical to that of the polymer synthesized. For example, for the synthesis of cationic polyelectrolytes, a cationic dispersing polyelectrolyte is generally used.

Examples of the dispersants that may be mentioned include the cationic polyelectrolytes obtained by polymerization of 50 to 100 mol % of at least one cationic monomer chosen from the salts, such as the hydrochlorides and sulfates, of dimethylaminoethyl (meth)acrylate, of N-dimethylaminopropyl(meth)-acrylamide and of di(meth)allylamine, (meth)acryloyloxy-ethyltrimethylammonium chloride, (meth)acrylamido-propyltrimethylammonium chloride and dimethyldiallyl-ammonium chloride, and of 50 to 0 mol % of acrylamide. A polyamine such as a polyalkyleneamine may, for example, also be used.

The dispersant is, for example, used in an amount ranging from 1% to 10% by weight relative to the total weight of the monomers to be polymerized.

The saline aqueous solution that serves as synthesis and dispersion medium for the high molecular weight water-soluble polymer is a solution of at least one mineral salt chosen, for example, from divalent anionic salts. Examples of anionic salts. that may be mentioned include ammonium sulfate, ammonium hydrogen sulfate, sodium sulfate, sodium hydrogen sulfate, magnesium sulfate, ammonium sulfate, magnesium hydrogen sulfate, aluminium sulfate and aluminium hydrogen sulfate. In one embodiment, at least one of ammonium sulfate and sodium sulfate is used.

The concentration of the at least one salt should, for example, be sufficient to induce the precipitation of the water-soluble polymer formed in the polymerization medium, and may be up to the saturation concentration of each salt. To obtain such a precipitation, the salt concentration is, for example, at least equal to 10% by weight, such as greater than 15% by weight and generally less than 50% by weight relative to the total weight of the polymer solution or dispersion. The saline aqueous solution may also comprise at least one monovalent salt, such as sodium chloride and ammonium chloride.

The heterogeneous free-radical polymerization in aqueous medium as described above is usually accompanied by a large increase in the viscosity of the reaction medium, which is reflected by difficulties in stirring, a lack of homogeneity of the reaction medium and an increase in the particle size of the polymer particles formed. To prevent such an increase in viscosity, it has been proposed, in European patent application EP 0 943 628, to add to the polymerization medium at least one agent for preventing the increase in viscosity of the reaction medium during polymerization.

The high molecular weight water-soluble polymers used herein are, for example, prepared in the presence of such an agent for preventing the increase in viscosity.

These agents for preventing the increase in viscosity of the reaction medium are chosen, for example, from:
(1) polycarboxylic acids and salts thereof,
(2) polyphenols,
(3) cyclic compounds comprising a hydroxyl group and a carboxyl group, and salts thereof,
(4) gluconic acid and salts thereof,
(5) the reaction products obtained by reacting a methoxyhydroquinone and/or a cationic (meth)acrylic monomer with a free-radical-generating compound, under an oxidizing atmosphere,
(6) the reaction products obtained by reacting a cationic (meth)acrylic polymer with a free-radical-generating compound, under an oxidizing atmosphere,
(7) the reaction products obtained by reacting a cationic (meth)acrylic polymer with an oxidizing agent,
   and mixtures thereof.

The addition of at least one agent for preventing the increase in viscosity as described above makes it possible to perform the polymerization of the water-soluble monomers described above with a low-power stirrer while at the same time avoiding the formation of coarse particles. The agents for preventing an increase in viscosity are, for example, soluble in the aqueous reaction medium.

Examples of compounds (A) that may be mentioned include oxalic acid, adipic acid, tartaric acid, malic acid and phthalic acid, and the salts thereof.

Examples of compounds (B) that may be mentioned include resorcinol and pyrogallol.

Examples of compounds (C) that may be mentioned include m-hydroxybenzoic acid, p-hydroxy-benzoic acid, salicylic acid, gallic acid and tannic acid, and the salts thereof.

Examples of compounds (D) that may be mentioned include sodium gluconate, potassium gluconate, ammonium gluconate and various amine salts of gluconic acid.

Examples of compounds (E) that may be mentioned include those obtained by reacting a free-radical-generating compound, under a stream of oxygenated gas, in a solution comprising at least one monomer chosen from methoxy-hydroquinone and cationic (meth)acrylic monomers. The free-radical-generating compound may be an initiator commonly used for free-radical polymerization. Examples that may be mentioned include water-soluble azo initiators such as 2,2′-azobis(2-amidinopropane) hydrochloride sold, for example, under the name V-50 by the company Wako Chemical Industries, 2,2′-azobis[2-(2-imidazolin-2-yl)propane] hydrochloride sold, for example, under the trade name VA-044 by the company Wako Chemical Industries, and an initiator chosen from the water-soluble redox agents, such as the ammonium persulfate/sodium hydrogen sulfite combination.

An agent for preventing an increase in viscosity (F) may be obtained by reacting a free-radical initiator, under an oxygenated atmosphere, with a dispersant as disclosed herein. The polymerization initiator may be a water-soluble azo initiator or a water-soluble redox agent as described above.

The compounds (G) may be obtained in the form of oxidized polymers of low molecular mass by oxidation of a cationic dispersant as disclosed herein obtained by polymerization of a cationic (meth)acrylic monomer, using hydrogen peroxide or a halogen as oxidizing agent.

As cationic (meth)acrylic monomers used for the preparation of agents for preventing an increase in viscosity (E), (F) and (G), examples that may be mentioned include dimethylaminoethyl (meth)acrylate hydrochloride or sulfate, (meth)acryloyloxyethyl-trimethylammonium chloride, (meth)acryloyloxyethyl-dimethylbenzylammonium chloride, the hydrochloride or sulfate derived from N-dimethylaminopropyl(meth)-acrylamide, (meth)acrylamidopropyltrimethylammonium chloride, dimethylaminohydroxypropyl (meth)acrylate chloride or sulfate, (meth)acryloyloxyhydroxypropyl-trimethylammonium chloride and (meth)acryloyloxy-hydroxypropyldimethylbenzylammonium chloride.

These agents for preventing an increase in viscosity (A) to (G) may be used alone or as a mixture, in an amount ranging, for example, from 10 ppm to 10 000 ppm relative to the total weight of the reaction solution.

The water-soluble monomers polymerized by heterogeneous free-radical polymerization to obtain high molecular weight water-soluble polymers are monomers comprising at least one ethylenic double bond chosen, for example, from vinyl, acrylic and allylic double bonds. They may be cationic, anionic or nonionic and may be used as a mixture, wherein at least one monomer is cationic.

Examples of water-soluble anionic monomers that may be mentioned include acrylic acid, methacrylic acid, acrylamido-2-methylpropanesulfonic acid and itaconic acid. These anionic monomers are at least partially neutralized in the form of a salt of an alkali metal (for example sodium and potassium), of an alkaline-earth metal, of ammonium or of an organic amine such as an alkanolamine, for example, ethanolamine.

Water-soluble nonionic monomers that may be mentioned include, for example, acrylamide, methacrylamide, N-vinylformamide, N-vinylacetonamide, hydroxypropyl acrylate and hydroxypropyl methacrylate.

The water-soluble cationic monomers are, for example, chosen from di(C₁₋₄ alkyl)diallylammonium salts and the compounds of formula (I) 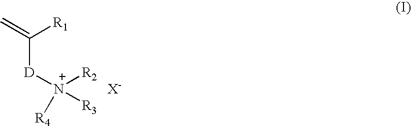
wherein

R₁ is chosen from a hydrogen atom and a methyl group,

R₂ and R₃, which may be identical or different, are each chosen from a hydrogen atom and linear and branched C₁₋₄ alkyl groups,

R₄ is chosen from a hydrogen atom, linear and branched C₁₋₄ alkyl groups, and an aryl group,

D is the following unit 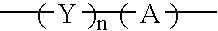
wherein Y is chosen from amide (—CO—NH—), ester (—O—CO— or —CO—O—), urethane (—O—CO—NH—) and urea (—NH—CO—NH—) functional groups,

A is chosen from linear, branched and cyclic C₁₋₁₀ alkylene groups, which may be substituted or interrupted with at least one ring chosen from divalent aromatic and heteroaromatic rings, or which may be interrupted with at least one hetero atom chosen from O, N, S and P, and which may comprise at least one functional group chosen from ketone, amide, ester, urethane and urea functional groups,
n is 0 or 1, and
X is an anionic counterion such as chloride and sulfate ions.

Examples of water-soluble cationic monomers that may be mentioned include dimethylaminoethyl (meth)acrylate hydrochloride or sulfate, (meth)acryloyloxyethyltrimethylammonium chloride, (meth)acryloyloxyethyldimethylbenzylammonium chloride, N-dimethylaminopropyl(meth)acrylamide hydrochloride or sulfate, (meth )acrylamidopropyltrimethylammonium chloride, (meth)acrylamidopropyldimethylbenzylammonium chloride, dimethylaminohydroxypropyl (meth)acrylate hydrochloride or sulfate, (meth)acryloyloxyhydroxy-propyltrimethylammonium chloride, (meth )acryloyloxy-hydroxypropyldimethylbenzylammonium chloride and dimethyldiallylammonium chloride.

In one embodiment, the high molecular weight water-soluble polymer is obtained by heterogeneous free-radical polymerization of a monomer mixture comprising from 0 to 30 mol % of acrylic acid, from 0 to 95.5 mol % of acrylamide and from 0.5 to 100 mol % of at least one cationic monomer of formula (I).

In another embodiment, the drawing polymer is obtained by heterogeneous free-radical polymerization of a monomer mixture comprising from 0 to 95.5 mol % of acrylamide and from 4.5 to 100 mol % of at least one cationic monomer of formula (I).

In yet another embodiment, the water-soluble polymers are obtained by polymerization of a monomer mixture comprising acrylic acid and at least one cationic monomer of formula (I), in which the number of moles of the at least one cationic monomer of formula (I) is greater than the number of moles of acrylic acid.

Water-soluble polyelectrolytes that may be used herein are chosen, for example, from those polymerized using monomer mixtures comprising, respectively:
1. 10 mol % of acryloyloxyethyldimethylbenzyl-ammonium chloride and 90 mol % of acrylamide;
2. 30 mol % of acryloyloxytrimethylammonium chloride, 50 mol % of acryloxyethyldimethylbenzyl-ammonium chloride and 20 mol % of acrylamide;
3. 10 mol % of acryloyloxyethyltrimethylammonium chloride and 90 mol % of acrylamide;
4. 30 mol % of diallyldimethylammonium chloride and 70 mol % of acrylamide.

The drawing polymers, such as the water-soluble polymers, used herein, have a weight-average molecular mass of greater than 1 000 000, such as ranging from 1 000 000 to 50 000 000. This weight-average molecular mass is determined via the RSV (Reduced Specific Viscosity) method as defined in “Principles of Polymer Chemistry”, Cornell University Press, Ithaca, N.Y., 1953, chapter VII entitled “Determination of Molecular Weight”, pages 266-316.

The concentration of particles of the at least one water-soluble polymer as a dispersion in a saline aqueous solution in (a1) ranges, for example, from 0.01% to 20% by weight, relative to the total weight of the dispersion. The concentration of the at least one water-soluble polymer as a solution in (a2) ranges, for example, from 0.01% to 20% by weight, relative to the total weight of the solution.

The concentration of the high molecular weight water-soluble polymer dispersion or solution is, for example, chosen such that the concentration of the water-soluble polymer ranges from 0.01% to 10% by weight such as from 0.05% to 5% by weight relative to the total weight of the final composition.

The concentration of the at least one drawing polymer ranges, for example, from 0.01% to 10% by weight such as from 0.05% to 5% by weight relative to the total weight of the final composition.

The at least one detergent surfactant is chosen from anionic, amphoteric, nonionic and zwitterionic surfactants.

As disclosed herein, the at least one detergent surfactant is present in an amount ranging from 4% to 50% by weight, such as from 6% to 30% by weight and further such as from 8% to 25% by weight, relative to the total weight of the final composition.

The surfactants that are suitable to be used herein are chosen, for example, from the following:

(i) Anionic Surfactant(s):

In the context of the present disclosure, their nature is not a truly critical feature.

Thus, as examples of anionic surfactants that may be used, alone or as mixtures, in the context of the present disclosure, mention may be made in a non-limiting manner of salts (for example, alkaline salts, such as sodium salts, ammonium salts, amine salts, amino alcohol salts and magnesium salts) of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, wherein the alkyl or acyl radical of all of these various compounds comprises from 12 to 20 carbon atoms, and the aryl radical is chosen, for example, from phenyl and benzyl groups. Among the anionic surfactants that may also be used, mention may also be made of fatty acid salts such as the salts of oleic, ricinoleic, palmitic and stearic acids, coconut oil acid or hydrogenated coconut oil acid; acyl lactylates wherein the acyl radical comprises from 8 to 20 carbon atoms. Use may also be made of weakly anionic surfactants, such as alkyl-D-galactosideuronic acids and their salts, and also polyoxyalkylenated carboxylic ether acids and their salts, for example, those comprising from 2 to 50 ethylene oxide groups, and mixtures thereof. Anionic surfactants of the polyoxyalkylenated carboxylic ether acid or salt type are, for example, those that correspond to formula (1) below:
R₁—(OC₂H₄)ₙ—OCH₂COOA (1)
wherein:

R₁ is chosen from alkyl, alkylamido and alkaryl groups, and n is an integer or decimal number (average value) that may range from 2 to 24 such as from 3 to 10, wherein the alkyl radical comprises from 6 to 20 carbon atoms, and the aryl radical is, for example, phenyl.

A is chosen from H, ammonium, Na, K, Li, Mg and monoethanolamine and triethanolamine residues. Mixtures of compounds of formula (1) can also be used, such as mixtures in which the groups R₁ are different.

Compounds of formula (1) are sold, for example, by the company Chem Y under the name Akypo (NP40, NP70, OP40, OP80, RLM25, RLM38, RLMQ 38 NV, RLM 45, RLM 45 NV, RLM 100, RLM 100 NV, RO 20, RO 90, RCS 60, RS 60, RS 100, RO 50) or by the company Sandoz under the name Sandopan (DTC Acid, DTC).

(ii) Nonionic Surfactant(s):

Nonionic surfactants are likewise compounds that are well known per se (see, for example, in this respect “Handbook of Surfactants” by M. R. Porter, published by Blackie & Son (Glasgow and London), 1991, pp. 116-178) and, in the context of the present disclosure, their nature is not a critical feature. Thus, they can be chosen, for example, from (non-limiting list) polyethoxylated, polypropoxylated and polyglycerolated fatty alcohols, polyethoxylated, polypropoxylated and polyglycerolated α-diols, polyethoxylated, polypropoxylated and polyglycerolated alkylphenols and polyethoxylated, polypropoxylated and polyglycerolated fatty acids, all having a fatty chain comprising, for example, from 8 to 18 carbon atoms, wherein it is possible for the number of ethylene oxide or propylene oxide groups to range, for example, from 2 to 50 and for the number of glycerol groups to range, for example, from 2 to 30. Mention may also be made of copolymers of ethylene oxide and of propylene oxide, condensates of ethylene oxide and of propylene oxide with fatty alcohols; polyethoxylated fatty amides having, for example, from 2 to 30 mol of ethylene oxide, polyglycerolated fatty amides comprising on average from 1 to 5, such as from 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides and N-acylaminopropylmorpholine oxides. It will be noted that the nonionic surfactants chosen, for example, from alkylpolyglycosides are suitable in the context of the present disclosure.

(iii) Amphoteric or Zwitterionic Surfactant(s):

The amphoteric or zwitterionic surfactants, whose nature is not a critical feature in the context of the present disclosure, can be chosen, for example, from (non-limiting list) aliphatic secondary and tertiary amine derivatives in which the aliphatic radical is chosen from linear and branched chains comprising from 8 to 18 carbon atoms and comprising at least one water-soluble anionic group (for example, carboxylate, sulfonate, sulfate, phosphate and phosphonate); mention may also be made, for example, of (C₈-C₂₀)-alkylbetaines, sulfobetaines, (C₈-C₂₀)alkylamido(C₁-C₆)alkylbetaines and (C₈-C₂₀)alkylamido(C₁-C₆)alkylsulfobetaines.

Among the amine derivatives, mention may be made, for example, of the products sold under the name Miranol, as described in U.S. Pat. Nos. 2,528,378 and 2,781,354 and classified in the CTFA dictionary, 3rd edition, 1982, under the names Amphocarboxyglycinates and Amphocarboxypropionates, with the respective structures:
R₂—CONHCH₂CH₂—N⁺(R₃)(R₄)(CH₂COO⁻) (2)
wherein: R₂ is chosen from an alkyl radical of an acid R₂—COOH present in hydrolyzed coconut oil, and heptyl, nonyl and undecyl radicals, R₃ is a β-hydroxyethyl group and R₄ is a carboxymethyl group;
and
R₂—CONHCH₂CH₂—N(B)(C) (3)
wherein:
B is —CH₂CH₂OX′, C is —(CH₂)_{z}—Y′, with z=1 or 2,
X′ is chosen from a —CH₂CH₂—COOH group and a hydrogen atom,
Y′ is chosen from —COOH and —CH₂—CHOH—SO₃H radicals,
R₂ is chosen from an alkyl radical of an acid R₂—COOH present in coconut oil or in hydrolyzed linseed oil, alkyl radicals, such as C₇, C₉, C₁₁ and C₁₃ alkyl radicals, a C₁₇ alkyl radical and its iso form, and an unsaturated C₁₇ radical.

For example, mention may be made of the cocoamphocarboxyglycinate sold under the trade name Miranol C2M concentrate by the company Miranol.

Mixtures of surfactants such as mixtures of anionic surfactants, mixtures of anionic surfactants and of amphoteric, cationic or nonionic surfactants, or mixtures of cationic surfactants with nonionic or amphoteric surfactants, may be used in the compositions as disclosed herein. A mixture comprising at least one anionic surfactant and at least one amphoteric surfactant is, for example, used.

The amount of anionic surfactants ranges, for example, from 4% to 50% by weight relative to the total weight of the cosmetic composition, such as from 5% to 35% by weight and further such as from 8% to 25% by weight relative to the total weight of the cosmetic composition.

The amount of amphoteric and/or nonionic surfactants, when they are present, ranges, for example, from 0.5% to 20% by weight such as from 1% to 15% by weight, relative to the total weight of the composition.

The particles as disclosed herein may be mineral or organic. The particles are chosen, for example, from glass particles, polyamide particles such as Nylon, and optionally expanded polymers or copolymers of acrylonitrile, of vinylidene chloride, of vinyl chloride and/or of acrylic or styrene monomer. The acrylic monomer is, for example, a methyl or ethyl acrylate or methacrylate. The styrene monomer is, for example, α-methylstyrene or styrene.

The mineral particles comprise, for example, at least one element chosen from columns IIa, IIIa and IVa, IIIb and IVb of the Periodic Table of the Elements, such as from columns IIa and IVa. They are chosen, for example, from particles comprising at least 10% by weight of calcium carbonate or of at least one silicate. Particles comprising at least 90% of aluminium oxide, silicas, magnesium oxide, zinc oxides, titanium oxides and barium sulfate, and mixtures thereof may also be used.

The solid mineral particles have, for example, a number-average primary size ranging from 2 nm to 2 microns, such as from 5 nm to 500 nm and further such as from 10 nm to 250 nm.

The particles as disclosed herein may, for example, be in any form, for example in the form of spheres, flakes, needles, platelets or totally random forms. In one embodiment, the particles are substantially spherical.

As used herein, the term “primary particle size” means the maximum size that it is possible to measure between two diametrically opposite points on an individual particle. The size may be determined, for example, by transmission electron microscopy or from the measurement of the specific surface area via the BET method, or alternatively by means of a laser granulometer.

In accordance with the present disclosure, the particle may be a solid particle formed entirely from calcium carbonate. Calcium carbonate may also totally or partially constitute the core of the particle, this core being covered with another constituent, for instance an oxide, a silicate or a metal. Calcium carbonate may also exclusively form the coating of a substrate of different chemical constitution, for instance an oxide, a silicate or a metal.

In the case where the particles are formed from calcium carbonate and from other fillers, the calcium carbonate is in free form and does not form chemical bonds with the other fillers. It is then a case of an alloy between the calcium carbonate and other fillers, such as with metal or metalloid oxides, obtained, for example, by melting of these various constituents.

When the particles comprising at least 10% by weight of calcium carbonate also comprise a metal or metalloid oxide, this oxide is, for example, chosen from silicon oxide, boron oxide and aluminium oxide.

In one embodiment, the particles comprise at least 50% by weight such as at least 70% by weight of calcium carbonate. The particles comprising more than 90% by weight of calcium carbonate are, for example, used according to the present disclosure.

In another embodiment, the particles comprising at least 10% by weight of calcium carbonate are substantially pure calcium carbonate particles.

The calcium carbonate that is suitable in the compositions as disclosed herein may be of natural origin or may be of synthetic origin. When the calcium carbonate is of synthetic origin, it may be obtained from calcium oxide, calcium peroxide, calcium acetate or calcium ethoxide.

The aluminium oxide particles according to the present disclosure comprise any optionally hydrated alumina, for instance boehmite.

The silicates that may be used according to the present disclosure may be chosen from sodium, magnesium and lithium silicates. The compounds sold by the company Laporte under the names Laponite XLG and Laponite XLS may, for example, be used.

When the particles comprising at least 10% by weight of at least one silicate also comprise a metal or metalloid oxide, this oxide is, for example, chosen from silicon oxide, boron oxide and aluminium oxide.

The silicates that are suitable in the compositions as disclosed herein may be of natural origin or of synthetic origin.

As glass particles that may be used herein, mention may be made of the hollow glass beads sold by the company 3M under the reference Scotchlite Glass Bubbles S22. 95% of these beads have a diameter of less than 74 μm.

Nylon particles that may be used are, for example, the “Orgasol” particles sold by the company Atochem. These particles are porous solid spheres with a diameter ranging, for example, from 5 μm to 60 μm.

In one embodiment, the particles are deformable hollow particles of an expanded copolymer of vinylidene chloride and of acrylonitrile or of vinylidene chloride, of acrylonitrile and of methacrylate. It is possible, for example, to use a terpolymer comprising: from 0% to 60% of units derived from vinylidene chloride, from 20% to 90% of units derived from acrylonitrile and from 0% to 50% of units derived from an acrylic or styrene monomer, the sum of the percentages (by weight) being equal to 100. These particles may be dry or hydrated and are, for example, those sold under the brand name Expancel by the company Nobel Casco, such as under the references 551 DE 12 (particle size of about 12 μm and density of about 40), 551 DE 20 (particle size of about 30 μm and density of about 65), 551 DE 50 (particle size of about 40 μm), 461 DE 50 and 642 WE 50 with a particle size of about 50 μm, and 551 DE 80 (particle size of about 80 μm).

It is also possible to use particles of this same terpolymer having a particle size of about 18 μm and a density of about 60 kg/m³ to 80 kg/m³, or alternatively with a particle size of about 34 μm and a density of about 20.

It is also possible to use particles of vinylidene chloride and of acrylonitrile polymer or of vinylidene chloride, of acrylonitrile and of non-expanded methacrylate polymer, for instance the products sold under the brand name Expancel with the references 551 DU 10 (particle size of about 10 μm) and 461 DU 15 (particle size of about 15 μm).

As other polymer particles that may be used herein, mention may also be made of the polymers and copolymers obtained from itaconic, citraconic, maleic and fumaric acids or esters, or from vinyl acetate or lactate. See in this respect document JP-A-2-112 304.

As used herein, the insoluble particles may be present in an amount ranging, for example, from 0.001% to 20% by weight, such as from 0.01% to 10% by weight and further such as from 0.1% to 3% by weight relative to the total weight of the final composition.

In one embodiment, the compositions may also comprise at least one cationic polymer other than the drawing polymer as disclosed herein.

The at least one cationic polymer that may be used herein may be chosen from all those already known per se as improving the cosmetic properties of the hair, for example, those described in European patent application EP-A-0 337 354 and in French patent applications FR-A-2 270 846; 2 383 660; 2 598 611; 2 470 596; and 2 519 863, and having a suitable cationic charge density.

Even more generally, as used herein, the term “cationic polymer” means any polymer comprising groups chosen from at least one of cationic groups and groups that may be ionized into cationic groups.

The cationic polymers are chosen, for example, from those comprising units comprising at least one amine group chosen from primary, secondary, tertiary and quaternary amine groups that either may form part of the main polymer chain or may be borne by a side substituent directly attached thereto.

The cationic polymers used generally have a number-average or weight-average molar mass ranging, for example, from 500 to 5×10⁶ such as from 10³ to 3×10⁶.

Among the cationic polymers that may be mentioned, examples include polymers of the polyamine, polyamino amide and polyquaternary ammonium type. These are known products.

The polymers of the polyamine, polyamino amide and polyquaternary ammonium type that may be used herein, and that may, for example, be mentioned, are those described in French patents Nos. 2 505 348 and 2 542 997. Among these polymers, mention may be made, for example, of:
(1) homopolymers or copolymers derived from acrylic or methacrylic esters or amides and comprising at least one of the units of the following formulae: 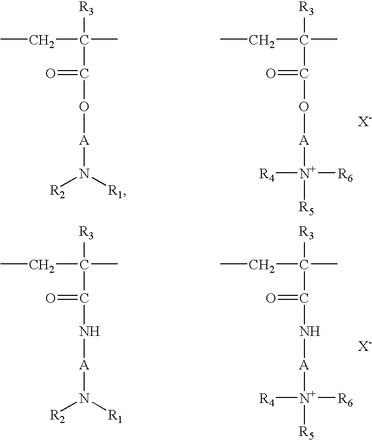
   wherein:
R₃, which may be identical or different, are chosen from a hydrogen atom and a CH₃ radical;
A, which may be identical or different, are chosen from linear and branched alkyl groups comprising from 1 to 6 carbon atoms, such as 2 and 3 carbon atoms, and hydroxyalkyl groups comprising from 1 to 4 carbon atoms;
R₄, R₅ and R₆, which may be identical or different, are each chosen from alkyl groups comprising from 1 to 18 carbon atoms, such as from 1 to 6 carbon atoms and a benzyl radical;
R₁ and R₂, which may be identical or different, are each chosen from hydrogen and alkyl groups comprising from 1 to 6 carbon atoms, such as methyl or ethyl;
X⁻ is an anion derived from a mineral or organic acid, such as a methosulfate anion or a halide such as chloride or bromide.

The copolymers of family (1) can also comprise at least one unit derived from comonomers that may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone and vinylcaprolactam, and vinyl esters.

Thus, among these copolymers of family (1), mention may be made, for example, of:
copolymers of acrylamide and of dimethylaminoethyl methacrylate quaternized with dimethyl sulfate or with a dimethyl halide, such as the product sold under the name Hercofloc by the company Hercules,
the copolymers of acrylamide and of methacryloyloxyethyltrimethylammonium chloride described, for example, in European patent application EP-A-080 976 and sold under the name Bina Quat P 100 by the company Ciba Geigy,
the copolymer of acrylamide and of methacryloyloxyethyltrimethylammonium methosulfate sold under the name Reten by the company Hercules,
quaternized or non-quaternized vinylpyrrolidone/dialkylaminoalkyl acrylate or methacrylate copolymers. These polymers are described in detail in French Patent Nos. 2 077 143 and 2 393 573,
dimethylaminoethyl methacrylate/vinylcaprolactam/vinylpyrrolidone terpolymers,
vinylpyrrolidone/methacrylamidopropyldimethylamine copolymers, and
quaternized vinylpyrrolidone/dimethylaminopropylmethacrylamide copolymers.
(2) cationic polysaccharides, such as cationic celluloses and cationic galactomannan gums. Among the cationic polysaccharides that may be mentioned, examples include cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are described, for example, in French Patent No. 1 492 597. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described, for example, in U.S. Pat. No. 4,131,576 such as hydroxyalkylcelluloses, for instance hydroxymethyl-, hydroxyethyl- and hydroxypropylcelluloses grafted, for example, with a methacryloyl-ethyltrimethylammonium, methacrylamidopropyltrimethylammonium or dimethyl-diallylammonium salt.

The cationic galactomannan gums are described, for example, in U.S. Pat. Nos. 3,589,578 and 4,031,307, such as guar gums comprising trialkylammonium cationic groups. In one embodiment, guar gums modified with a salt (e.g., chloride) of 2,3-epoxypropyltrimethylammonium may be used.
(3) polymers comprising piperazinyl units and divalent alkylene or hydroxyalkylene radicals comprising at least one chain chosen from straight and branched chains, optionally interrupted by at least one entity chosen from oxygen, sulfur and nitrogen atoms and aromatic and heterocyclic rings, and also the oxidation and/or quaternization products of these polymers. Such polymers are described, for example, in French Patent Nos. 2 162 025 and 2 280 361.
(4) water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound that is reactive with respect to a bis-halohydrin, a bis-azetidinium, a bis-haloacyldiamine, a bis-alkyl halide, an epihalohydrin, a diepoxide or a bis-unsaturated derivative; wherein the crosslinking agent is used in an amount ranging from 0.025 to 0.35 mol per amine group of the polyamino amide; these polyamino amides can be alkylated or, if they contain one or more tertiary amine functional groups, they can be quaternized. Such polymers are described, for example, in French Patent Nos. 2 252 840 and 2 368 508.
(5) polyamino amide derivatives resulting from the condensation of polyalkylene polyamines with polycarboxylic acids followed by alkylation with difunctional agents. Mention may be made, for example, of adipic acid/dialkylaminohydroxyalkyldialkylene-triamine polymers in which the alkyl radical comprises from 1 to 4 carbon atoms such as methyl, ethyl and propyl. Such polymers are described, for example, in French Patent No. 1 583 363.

Among these derivatives, mention may be made, for example, of the adipic acid/dimethylaminohydroxypropyl/diethylenetriamine polymers sold under the name “Cartaretine F, F4 or F8” by the company Sandoz.
(6) polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms. The molar ratio between the polyalkylene polyamine and the dicarboxylic acid ranges from 0.8:1 to 1.4:1; the polyamino amide resulting therefrom is reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.

Polymers of this type are sold, for example, under the name “Hercosett 57” by the company Hercules Inc. and by the company Hercules in the case of the adipic acid/epoxypropyl/diethylenetriamine copolymer.
(7) cyclopolymers of alkyldiallylamine or of dialkyldiallylammonium, such as the homopolymers or copolymers comprising, as main constituent of the chain, units corresponding to formula (I) or (I′): 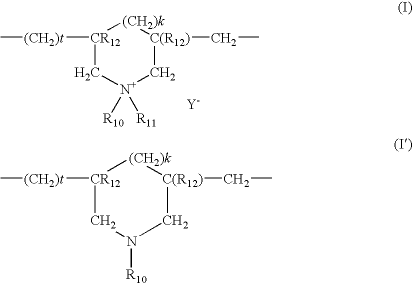
   wherein k and t, which may be identical or different, are equal to 0 or 1, the sum k+t being equal to 1; R₁₂ is chosen from a hydrogen atom and a methyl radical; R₁₀ and R₁₁, which may be identical or different, are each chosen from alkyl groups comprising from 1 to 6 carbon atoms, hydroxyalkyl groups wherein the alkyl group comprises, for example, from 1 to 5 carbon atoms, and lower (C₁-C₄) amidoalkyl groups. In one embodiment, R₁₀ and R₁₁ can form, together with the nitrogen atom to which they are attached, heterocyclic groups such as piperidyl and morpholinyl, Y⁻ is an anion such as bromide, chloride, acetate, borate, citrate, tartrate, bisulfate, bisulfite, sulfate and phosphate. These polymers are described, for example, in French Patent No. 2 080 759 and in its Certificate of Addition 2 190 406.

In one embodiment, R₁₀ and R₁₁, which may be identical or different, are each chosen, for example, from alkyl groups comprising from 1 to 4 carbon atoms.

Among the polymers defined above, mention may be made, for example, of the dimethyldiallylammonium chloride homopolymer sold under the name “Merquat 100” by the company Nalco (and its homologues of low weight-average molar mass) and copolymers of diallyldimethylammonium chloride and of acrylamide.
(8) diquaternary ammonium polymers comprising repeating units corresponding to the formula (II): 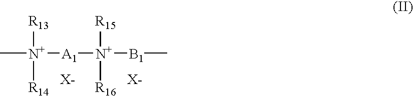
   wherein:

R₁₃, R₁₄, R₁₅ and R₁₆, which may be identical or different, are each chosen from aliphatic, alicyclic and arylaliphatic radicals comprising from 1 to 20 carbon atoms and lower hydroxyalkylaliphatic radicals. Alternatively R₁₃, R₁₄, R₁₅ and R₁₆, together or separately, form, with the nitrogen atoms to which they are attached, heterocycles optionally comprising a second hetero atom other than nitrogen. In one embodiment, R₁₃, R₁₄ R₁₅ and R₁₆ are chosen from linear and branched C₁-C₆ alkyl radicals substituted with at least one group chosen from nitrile, ester, acyl and amide groups and groups —CO—O—R₁₇—D and —CO—NH—R₁₇—D wherein R₁₇ is an alkylene and D is a quaternary ammonium group;

A₁ and B₁, which may be identical or different, are each chosen from polymethylene groups comprising from 2 to 20 carbon atoms, which may be linear or branched, saturated or unsaturated, and which may comprise, linked to or intercalated in the main chain, at least one entity chosen from aromatic rings, and oxygen and sulfur atoms and sulfoxide, sulfone, disulfide, amino, alkylamino, hydroxyl, quaternary ammonium, ureido, amide and ester groups, and

X⁻ is an anion derived from a mineral or organic acid;

A₁, R₁₃ and R₁₅ can form, together with the two nitrogen atoms to which they are attached, a piperazine ring; in addition, if A₁ is chosen from linear and branched, saturated and unsaturated alkylene and hydroxyalkylene radicals, B₁ can also be a group (CH₂)ₙ—CO-D-OC—(CH₂)ₙ—;

wherein D is chosen from:

a) a glycol residue of formula: —O-Z-O—, wherein Z is chosen from linear and branched hydrocarbon-based radicals and a group corresponding to one of the following formulae:
—(CH₂—CH₂—O)ₓ—CH₂—CH₂—
—[CH₂—CH(CH₃)—O]_{y}—CH₂—CH(CH₃)—
wherein x and y, which may be identical or different, are each an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, wherein Y is chosen from linear and branched hydrocarbon-based radicals, or alternatively the divalent radical
—CH₂—CH₂—S—S—CH₂—CH₂—;

d) a ureylene group of formula: —NH—CO—NH—;

In one embodiment, X⁻ is an anion such as chloride or bromide.

These polymers generally have a number-average molar mass ranging, for example, from 1000 to 100 000.

Polymers of this type are described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is also, for example, possible to use polymers that comprise repeating units corresponding to the formula: 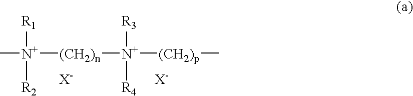
wherein R₁, R₂, R₃ and R₄, which may be identical or different, are each chosen from alkyl and hydroxyalkyl radicals comprising from 1 to 4 carbon atoms, n and p, which may be identical or different, are each an integer ranging from 2 to 20, and X⁻ is an anion derived from a mineral or organic acid.

One compound of formula (a) which may be used, for example, is the one for which R₁, R₂, R₃ and R₄ are each a methyl radical and n=3, p=6 and X═Cl, which is known as Hexadimethrine chloride according to the INCl (CTFA) nomenclature.
(9) polyquaternary ammonium polymers comprising units of formula (III): 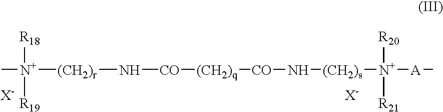
   wherein:

R₁₈, R₁₉, R₂₀ and R₂₁, which may be identical or different, are each chosen from a hydrogen atom and methyl, ethyl, propyl, β-hydroxyethyl, β-hydroxypropyl and —CH₂CH₂(OCH₂CH₂)ₚOH radicals,

wherein p is equal to 0 or to an integer ranging from 1 to 6, with the proviso that R₁₈, R₁₉, R₂₀ and R₂₁ are not simultaneously a hydrogen atom,

r and s, which may be identical or different, are each an integer ranging from 1 to 6,

q is equal to 0 or to an integer ranging from 1 to 34,

X⁻ is an anion such as a halide,

A is a divalent radical such as —CH₂—CH₂—O—CH₂—CH₂—.

Such compounds are described, for example, in patent application EP-A-122 324.

Among these products, mention may be made, for example, of the products “Mirapol® A 15”, “Mirapol® AD1”, “Mirapol® AZ1” and “Mirapol® 175” sold by the company Miranol.
(10) quaternary polymers of vinylpyrrolidone and of vinylimidazole, for instance the products sold under the names Luviquat® FC 905, FC 550 and FC 370 by the company BASF.
(11) crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, wherein the homo- or copolymerization is followed by crosslinking with a compound comprising at least one olefinic unsaturation, such as methylenebisacrylamide.

Other cationic polymers that may be used herein are cationic proteins or cationic protein hydrolysates, polyalkyleneimines, such as polyethyleneimines, polymers comprising at least one unit chosen from vinylpyridine and vinylpyridinium units, condensates of polyamines and of epichlorohydrin, polyquaternary ureylenes and chitin derivatives, such as chitosans and salts thereof;

the salts that may be used are, for example, chitosan acetate, lactate, glutamate, gluconate and pyrrolidonecarboxylate.

Among these compounds, mention may be made, for example, of chitosan with a degree of deacetylation of 90% by weight, and the chitosan pyrrolidonecarboxylate sold under the name Kytamer® PC by the company Amerchol.

Among all the cationic polymers that may be used herein, cationic cyclopolymers may, for example, be used, such as the dimethyldiallylammonium chloride homopolymers and copolymers sold under the names “Merquat 100”, “Merquat 550” and “Merquat S” by the company Nalco, quaternary polymers of vinylpyrrolidone and of vinylimidazole, crosslinked homopolymers or copolymers of methacryloyloxy(C₁-C₄)alkyltri(C₁-C₄)alkylammonium salts, and the chitosan pyrrolidonecarboxylate sold under the name Kytamer® PC by the company Amerchol, and mixtures thereof.

The amphoteric polymers that may be used herein may be chosen from polymers comprising units K and M randomly distributed in the polymer chain, wherein K is a unit derived from a monomer comprising at least one basic nitrogen atom and M is a unit derived from an acidic monomer comprising at least one group chosen from carboxylic and sulfonic groups, or alternatively K and M, which may be identical or different, may be chosen from groups derived from zwitterionic carboxybetaine or sulfobetaine monomers;

K and M may also be chosen from cationic polymer chains comprising at least one amine group chosen from primary, secondary, tertiary and quaternary amine groups, wherein at least one of the amine groups bears at least one group chosen from carboxylic and sulfonic groups linked. via a hydrocarbon-based radical. Alternatively, K and M form part of a chain of a polymer comprising an α,β-dicarboxylic ethylene unit in which one of the carboxylic groups has been made to react with a polyamine comprising at least one amine group chosen from primary and secondary amine groups.

The amphoteric polymers corresponding to the above definition are chosen, for example, from the following polymers:

(1) polymers resulting from the copolymerization of a monomer derived from a vinyl compound bearing a carboxylic group such as acrylic acid, methacrylic acid, maleic acid, and α-chloroacrylic acid, and a basic monomer derived from a substituted vinyl compound comprising at least one basic atom, such as dialkylaminoalkyl methacrylate and acrylate, dialkylaminoalkylmethacrylamide and -acrylamide. Such compounds are described, for example, in U.S. Pat. No. 3,836,537. Mention may also be made, for example, of the sodium acrylate/acrylamidopropyltrimethylammonium chloride copolymer sold under the name Polyquart KE 3033 by the company Cognis.

The vinyl compound may also be a dialkyldiallylammonium salt such as dimethyldiallylammonium salt (for example, chloride). The copolymers of acrylic acid and of the basic monomer are sold, for example, under the names Merquat 280 and Merquat 295 by the company Nalco.

(2) Polymers comprising units derived from:

a) at least one monomer chosen from acrylamides and methacrylamides substituted on the nitrogen with an alkyl radical,

b) at least one acidic comonomer comprising at least one reactive carboxylic group, and

c) at least one basic comonomer such as esters comprising primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that are used herein include, for example, groups in which the alkyl radicals comprise from 2 to 12 carbon atoms such as N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen, for example, from acrylic acid, methacrylic acid, crotonic acid, itaconic acid, maleic acid and fumaric acid and alkyl monoesters, comprising from 1 to 4 carbon atoms, of maleic or fumaric acids or anhydrides.

Examples of the basic comonomers include aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer may, for example, be used.

(3) Polyamino amides that are crosslinked and alkylated partially or totally derived from polyamino amides of general formula: [symbol]
CO—R₄—CO-Z [symbol] (IV)
wherein R₄ is a divalent radical derived from a saturated dicarboxylic acid, a mono- or dicarboxylic aliphatic acid comprising an ethylenic double bond, an ester of a lower alkanol, comprising from 1 to 6 carbon atoms of these acids or a radical derived from the addition of any one of the acids to a bis(primary) or bis(secondary) amine, and Z is chosen from bis(primary), mono- and bis(secondary) polyalkylene-polyamine radicals. In one embodiment, Z represents:

a) in proportions ranging from 60 to 100 mol %, the radical
—NH [symbol] (CH₂)ₓ—NH [symbol] ₚ (V)
wherein x=2 and p=2 or 3, or alternatively x=3 and p=2, this radical being derived from a compound chosen from diethylenetriamine, triethylenetetraamine, and dipropylenetriamine;

b) in proportions ranging from 0 to 40 mol %, the radical (V) above in which x=2 and p=1 and which is derived from ethylenediamine, or the radical derived from piperazine:

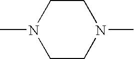

c) in proportions ranging from 0 to 20 mol %, the —NH—(CH₂)₆—NH— radical derived from hexamethylenediamine, these polyamino amines being crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are, for example, chosen from acids comprising from 6 to 10 carbon atoms, such as adipic acid, 2,2,4-trimethyladipic acid and 2,4,4-trimethyladipic acid, terephthalic acid and acids comprising an ethylenic double bond such as acrylic acid, methacrylic acid and itaconic acid.

The alkane sultones used in the alkylation are chosen, for example, from propane sultone and butane sultone, and the salts of the alkylating agents are chosen, for example, from the sodium and potassium salts.

(4) Polymers comprising zwitterionic units of formula: 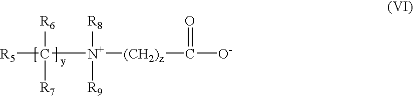
wherein R₅ is a polymerizable unsaturated group such as an acrylate, methacrylate, acrylamide or methacrylamide group, y and z, which may be identical or different, are each an integer from 1 to 3, R₆ and R₇, which may be identical or different, are each chosen from a hydrogen atom, methyl, ethyl and propyl, R₈ and R₉, which may be identical or different, are each chosen from a hydrogen atom and alkyl radicals such that the sum of the carbon atoms in R₈ and R₉ does not exceed 10.

The polymers comprising such units may also comprise units derived from non-zwitterionic monomers such as dimethyl or diethylaminoethyl acrylate or methacrylate, alkyl acrylates or methacrylates, acrylamides or methacrylamides, or vinyl acetate.

By way of example, mention may be made of the copolymer of butyl methacrylate/dimethylcarboxymethylammonioethyl methacrylate.

(5) Polymers derived from chitosan comprising monomer units corresponding to formulae (VII), (VIII) and (IX) below: 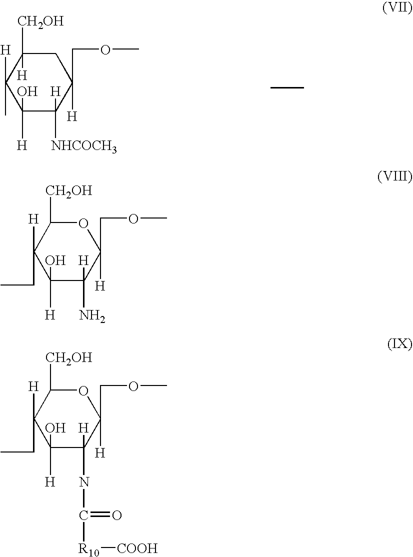
wherein the unit (VII) is present in a proportion ranging from 0 to 30%, the unit (VIII) in is present in a proportion ranging from 5 to 50% and the unit (IX) is present in a proportion ranging from 30 to 90%, it being understood that, in this unit (IX), R₁₀ is a radical of formula: 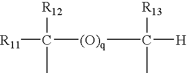
wherein q is equal to 0 or 1;
if q=0, R₁₁, R₁₂ and R₁₃, which may be identical or different, are each chosen from a hydrogen atom, methyl, hydroxyl, acetoxy and amino residues, monoalkylamine residues and dialkylamine residues that are optionally interrupted by at least one nitrogen atom and/or optionally substituted with at least one group chosen from amine, hydroxyl, carboxyl, alkylthio and sulfonic groups, alkylthio residues in which the alkyl group bears an amino residue, at least one of the radicals R₁₁, R₁₂ and R₁₃ being, in this case, a hydrogen atom;
or, if q=1, R₁₁, R₁₂ and R₁₃ are each chosen from a hydrogen atom, and also the base and acid addition salts thereof.

(6) Polymers derived from the N-carboxyalkylation of chitosan, such as N-carboxymethylchitosan and N-carboxybutylchitosan.

(7) Polymers corresponding to the general formula (X) such as those described, for example, in French Patent No. 1 400 366: 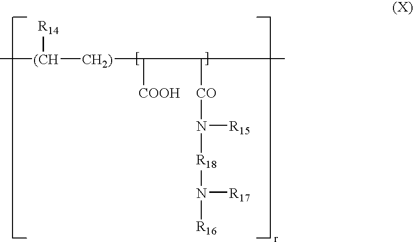
wherein R₁₄ is chosen from a hydrogen atom, and CH₃O, CH₃CH₂O and phenyl radicals, R₁₅ is chosen from hydrogen and lower alkyl radicals such as methyl and ethyl, R₁₆ is chosen from hydrogen and lower alkyl radicals such as methyl and ethyl, R₁₇ is chosen from lower alkyl radicals such as methyl and ethyl and a radical corresponding to the formula:
—R₁₈ N(R₁₆)₂, R₁₈ being chosen from —CH₂—CH₂—, —CH₂—CH₂—CH₂— and —CH₂—CH(CH₃)— groups, R₁₆ is chosen from hydrogen and lower alkyl radicals such as methyl and ethyl, and also the higher homologues of these radicals and comprising up to 6 carbon atoms, r is chosen such that the number-average molecular weight of the polymer ranges from 500 to 6,000,000, such as from 1,000 to 1,000,000.

(8) Amphoteric polymers of the type -D-X-D-X- chosen from:
a) polymers obtained by the action of chloroacetic acid or sodium chloroacetate on compounds comprising at least one unit of formula:
   -D-X-D-X-D- (XI)
   wherein D is a radical 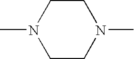
   and X is chosen from the symbols E and E′, wherein E or E′ which may be identical or different, are chosen from divalent alkylene radicals comprising at least one chain chosen from straight and branched chains comprising up to 7 carbon atoms in the main chain, wherein the divalent alkylene radicals are optionally substituted with at least one hydroxyl group. E or E′ can additionally comprise at least one atom chosen from oxygen, nitrogen and sulfur atoms, and 1 to 3 rings chosen from aromatic and heterocyclic rings. The oxygen, nitrogen and sulfur atoms can be present in the form of at least one group chosen from ether, thioether, sulfoxide, sulfone, sulfonium, alkylamine, alkenylamine, hydroxyl, benzylamine, amine oxide, quaternary ammonium, amide, imide, alcohol, ester and urethane groups;
b) polymers of formula:
   -D-X-D-X- (XII)
   wherein D is a radical 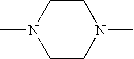
   and X is chosen from the symbols E and E′ and wherein at least one of X is E′; E having the meaning given above and E′ being chosen from divalent alkylene radicals comprising at least one chain chosen from straight and branched chains comprising up to 7 carbon atoms in the main chain, wherein the divalent alkylene radicals are optionally substituted with at least one hydroxyl radical. E′ can also comprise at least one nitrogen atom, substituted with an alkyl chain that is optionally interrupted by an oxygen atom, wherein the alkyl chain comprises at least one functional group chosen from carboxyl functional groups and hydroxyl functional groups and wherein the alkyl chain is betainized by reaction with a reactant chosen from chloroacetic acid and sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

In one embodiment, the amphoteric polymers of family (1) are used.

As disclosed herein, the cationic or amphoteric polymer(s) may be present in an amount ranging, for example, from 0.001% to 20% by weight, such as from 0.01% to 10% by weight and further such as from 0.02% to 5% by weight relative to the total weight of the final composition.

In one embodiment, the compositions may also comprise at least one silicone.

As silicones that may be used in the compositions as disclosed herein, mention may be made, for example, of volatile or non-volatile, cyclic or acyclic, branched or unbranched, organomodified or non-organomodified silicones, as described below.

The silicones that may be used herein may be soluble or insoluble in the composition and, for example, may be polyorganosiloxanes that are insoluble in the composition as disclosed herein; they may be in the form of oils, waxes, resins or gums.

As disclosed herein, all the silicones may be used in unmodified form or in the form of solutions, dispersions, emulsions, nanoemulsions or microemulsions.

The organopolysiloxanes are defined in greater detail in Walter Noll's “Chemistry and Technology of Silicones” (1968) Academic Press. They can be volatile or non-volatile.

When they are volatile, the silicones are, for example, chosen from those having a boiling point ranging from 60° C. to 260° C., such as those chosen from:
(i) cyclic silicones comprising from 3 to 7 such as from 4 to 5 silicon atoms. These are, for example, octamethylcyclotetrasiloxane sold, for example, under the name “Volatile Silicone 7207” by Union Carbide or “Silbione 70045 V 2” by Rhodia, decamethylcyclopentasiloxane sold under the name “Volatile Silicone 7158” by Union Carbide, and “Silbione 70045 V 5” by Rhodia, and mixtures thereof.

Mention may also be made of cyclocopolymers of the dimethylsiloxane/methylalkylsiloxane type, such as “Volatile Silicone FZ 3109” sold by the company Union Carbide, having the chemical structure:

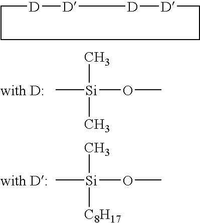

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;
(ii) linear volatile silicones comprising from 2 to 9 silicon atoms and having a viscosity of less than or equal to 5×10⁻⁶ m²/s at 25° C. An example is decamethyltetrasiloxane sold, for example, under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones may, for example, be used, such as polyalkylsiloxanes, polyarylsiloxanes, polyalkylarylsiloxanes, silicone gums, silicone resins, and polyorganosiloxanes modified with organofunctional groups, and also mixtures thereof. These silicones are, for example, chosen from polyalkylsiloxanes, among which mention may be made, for example, of polydimethylsiloxanes comprising trimethylsilyl end groups and having a viscosity of from 5×10⁻⁶ to 2.5 m²/s at 25° C. such as from 1×10⁻⁵ to 1 m²/s. The viscosity of the silicones is measured, for example, at 25° C. according to ASTM standard 445 Appendix C.

Among these polyalkylsiloxanes, mention may be made, in a non-limiting manner, of the following commercial products:
the Silbione® oils of the 47 and 70 047 series or the Mirasil® oils sold by Rhodia, such as the oil 70 047 V 500 000;
the oils of the Mirasil® series sold by the company Rhodia;
the oils of the 200 series from the company Dow Corning, such as DC200 with a viscosity of 60 000 mm²/s; and
the Viscasil® oils from General Electric and certain oils of the SF series (SF 96, SF 18) from General Electric.

Mention may also be made, for example, of polydimethylsiloxanes comprising dimethylsilanol end groups, known under the name dimethiconol (CTFA) such as the oils of the 48 series from the company Rhodia.

In this category of polyalkylsiloxanes, mention may also be made, for example, of the products sold under the names “Abil Wax® 9800 and 9801” by the company Goldschmidt, which are poly(C₁-C₂₀)alkylsiloxanes.

The polyalkylarylsiloxanes are, for example, chosen from polydimethyl/methylphenylsiloxanes, linear and branched polydimethyldiphenylsiloxanes with a viscosity of from 1×10⁻⁵ to 5×10⁻² m²/s at 25° C. Among these polyalkylarylsiloxanes, examples that may be mentioned include the products sold under the following names:
the Silbione® oils of the series 70 641 from Rhodia;
the oils of the Rhodorsil®70 633 and 763 series from Rhodia;
the oil Dow Corning 556 Cosmetic Grade Fluid from Dow Corning;
the silicones of the PK series from Bayer, for instance the product PK20;
the silicones of the PN and PH series from Bayer, for instance the products PN1000 and PH1000; and
certain oils of the SF series from General Electric, such as SF 1023, SF 1154, SF 1250 and SF 1265.

The silicone gums that may be used herein are, for example, polydiorganosiloxanes having a high number-average molecular weight ranging from 200 000 to 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, and mixtures thereof.

Mention may be made, for example, of the following products:
polydimethylsiloxane,
polydimethylsiloxane/methylvinylsiloxane gums,
polydimethylsiloxane/diphenylsiloxane,
polydimethylsiloxane/phenylmethylsiloxane, and
polydimethylsiloxane/diphenylsiloxane/methylvinylsiloxane.

Products that can be used herein are, for example, mixtures such as:
mixtures formed from a polydimethylsiloxane hydroxylated at the chain end, or dimethiconol (CTFA), and from a cyclic polydimethylsiloxane, also known as cyclomethicone (CTFA), such as the product Q2 1401 sold by Dow Corning; mixtures formed from a polydimethylsiloxane gum with a cyclic silicone, such as the product SF 1214 Silicone Fluid from General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs of different viscosities, such as of a PDMS gum and a PDMS oil, for example, the product SF 1236 from General Electric. The product SF 1236 is a mixture of an SE 30 gum defined above, having a viscosity of 20 m²/s, and an SF 96 oil, with a viscosity of 5×10⁻⁶ m²/s. This product comprises, for example, 15% SE 30 gum and 85% SF 96 oil.

The organopolysiloxane resins that can be used herein are, for example, crosslinked siloxane systems comprising the following units:
R₂SiO_{2/2}, R₃SiO_{1/2}, RSiO_{3/2} and SiO_{4/2} wherein R is chosen from hydrocarbon-based groups comprising from 1 to 16 carbon atoms and a phenyl group. Among these products, examples include those wherein R is chosen from C₁-C₄ lower alkyl groups, such as methyl, and a phenyl group.

Among these resins, mention may be made, for example, of the product sold under the name “Dow Corning 593” or those sold under the names “Silicone Fluid SS 4230 and SS 4267” by General Electric, which are silicones of dimethyl/trimethyl siloxane structure.

Mention may also be made, for example, of the trimethyl siloxysilicate type resins sold, for example, under the names X22-4914, X21-5034 and X21-5037 by the company Shin-Etsu.

The organomodified silicones that can be used herein are silicones as defined above and comprising in their structure at least one organofunctional group attached via a hydrocarbon-based group.

Among the organomodified silicones, mention may be made, for example, of polyorganosiloxanes comprising:

at least one group chosen from polyethyleneoxy and polypropyleneoxy groups optionally comprising at least one alkyl group chosen from C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are chosen, for example, from C₁-C₄ aminoalkyl groups. Trimethylsilyl amodimethicone and amodimethicones may, for example, be used;

thiol groups such as the products sold under the names “GP 72 A” and “GP 71” from Genesee;

alkoxylated groups such as the product sold under the name “Silicone Copolymer F-755” by SWS Silicones and Abil Wax® 2428, 2434 and 2440 by the company Goldschmidt;

hydroxylated groups such as the polyorganosiloxanes comprising a hydroxyalkyl functional group, described in French patent application FR-A-85/16334;

acyloxyalkyl groups such as the polyorganosiloxanes described in U.S. Pat. No. 4,957,732;

anionic groups of carboxylic type, such as in the products described in patent EP 186 507 from the company Chisso Corporation, or of alkylcarboxylic type, such as those present in the product X-22-3701 E from the company Shin-Etsu; 2-hydroxyalkyl sulfonate; 2-hydroxyalkyl thiosulfate such as the products sold by the company Goldschmidt under the names “Abil® S201” and “Abil® S255”;

hydroxyacylamino groups, such as the polyorganosiloxanes described in patent application EP 342 834. Mention may be made, for example, of the product Q2-8413 from the company Dow Corning.

The silicones as described above may be used alone or as a mixture, in an amount ranging, for example, from 0.01% to 20% by weight such as from 0.1% to 5% by weight relative to the total weight of the composition.

The term “cosmetically acceptable medium” means a medium that is compatible with keratin materials such as the skin, the eyelashes and the hair.

The cosmetically acceptable medium may comprise solely of water or of a mixture of water and of at least one cosmetically acceptable solvent chosen, for example, from C₁-C₄ lower alcohols, for instance ethanol, isopropanol, tert-butanol and n-butanol; alkylene glycols, for instance propylene glycol, and polyol ethers; and mixtures thereof.

In one embodiment, the composition comprises from 50% to 95% by weight of water relative to the total weight of the composition.

The compositions as disclosed herein have a final pH generally ranging from 3 to 10. For example, this pH ranges from 4.5 to 8. The pH may be adjusted to the desired value conventionally, by adding a base (organic or mineral base) to the composition, for example aqueous ammonia or a primary, secondary or tertiary (poly)amine, for instance monoethanolamine, diethanolamine, triethanolamine, isopropanolamine or 1,3-propanediamine, or alternatively by adding an acid, such as a carboxylic acid, for instance citric acid.

The compositions as disclosed herein may further comprise, in addition to the combination defined above, viscosity regulators such as electrolytes, or thickeners (associative or non-associative thickeners). Mention may be made, for example, of sodium chloride, sodium xylenesulfonate, scleroglucans, xanthan gums, fatty acid alkanolamides, alkyl ether carboxylic acid alkanolamides optionally oxyethylenated with up to 5 mol of ethylene oxide, such as the product sold under the name “Aminol A15” by the company Chem Y, crosslinked polyacrylic acids and acrylic acid copolymers such as crosslinked acrylic acid/C₁₀-C₃₀ alkyl acrylate copolymers. These viscosity regulators are used in the compositions as disclosed herein in an amount that may be up to 10% by weight relative to the total weight of the composition.

The compositions as disclosed herein may further comprise, for example, up to 5% of nacreous agents or opacifiers that are well known in the art, such as C₁₆ higher fatty alcohols, fatty-chain acyl derivatives such as ethylene glycol or polyethylene glycol monostearate or distearate, and fatty-chain (C₁₀-C₃₀) ethers such as distearyl ether and 1-(hexadecyloxy)-2-octadecanol.

The compositions as disclosed herein may optionally also comprise at least one additive chosen from foam synergists such as C₁₀-C₁₈ 1,2-alkanediols and fatty alkanolamides derived from monoethanolamine or diethanolamine, silicone or non-silicone sunscreens, anionic or nonionic polymers, cationic surfactants, proteins, protein hydrolysates, ceramides, pseudoceramides, linear or branched C₁₂-C₄₀ fatty acids such as 18-methyleicosanoic acid, hydroxy acids, vitamins, provitamins such as panthenol, animal, mineral or synthetic oils, and any other additive conventionally used in cosmetics that does not affect the properties of the compositions as disclosed herein.

The washing compositions as disclosed herein may also comprise any adjuvant usually encountered in the field of shampoos, for instance fragrances, preserving agents, sequestering agents, softeners, dyes, moisturizers, anti-dandruff agents or anti-seborrhoeic agents, and the like.

A person skilled in the art will take care to select this or these optional additional compound(s) and/or the amounts thereof such that the advantageous properties intrinsically associated with the combination as disclosed herein are not, or are not substantially, adversely affected by the envisioned addition(s).

These compositions may be in the form of optionally thickened liquids, creams or gels, and they are suitable, for example, for washing, optionally caring for and/or styling the hair.

The present disclosure also relates to a cosmetic process for treating keratin materials, comprising applying an effective amount of a composition as described above to the keratin materials, and rinsing after an optional leave-in time. In one embodiment, the composition may be used as a shampoo.

When the compositions as disclosed herein are used as standard shampoos, they are simply applied to wet hair and the lather generated by massaging or friction with the hands is then removed, after an optional action time, by rinsing with water, the operation possibly being repeated one or more times.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Concrete, but in no way limiting, examples illustrating the invention are as follows.

### EXAMPLES 1 to 4

Shampoo compositions in accordance with the invention were prepared:

| | 1 | 2 | 3 | 4 |
|---|---|---|---|---|
| Copolymer (90 mol/10 mol) of | — | 1% | — | 0.75% |
| acrylamide and of | | AM | | AM |
| dimethylaminoethyl acrylate | | | | |
| quaternized with benzyl chloride | | | | |
| (MW > 5 × 10⁶) as a | | | | |
| dispersion in a concentrated | | | | |
| saline aqueous solution | | | | |
| (Ultimer from Ondeo) | | | | |
| Copolymer (10 mol/90 mol) of | 1% | — | 1% | — |
| dimethylaminoethyl acrylate | AM | | AM | |
| quaternized with methyl chloride | | | | |
| and of acrylamide | | | | |
| (MW > 5 × 10⁶) as a | | | | |
| dispersion in a concentrated | | | | |
| saline aqueous solution | | | | |
| Sodium lauryl ether sulfate 2.2 EO | 12.5% | 12.5% | 12.5% | 12.5% |
| | AM | AM | AM | AM |
| Cocoamidopropylbetaine | 2.5% | 2.5% | 2.5% | 2.5% |
| | AM | AM | AM | AM |
| Calcium carbonate | 0.8% | 0.3% | 0.8% | — |
| (Omyapure 35 from Omya) | | | | |
| Chitosan PCA | 0.3% | — | 0.3% | 0.2% |
| (Kytamer PC from Amerchol) | | | | |
| Carnauba wax/synthetic wax | — | — | — | 0.3% |
| microbeads (Microcare 325 | | | | |
| from Micropowders) | | | | |
| Silver-coated glass flakes | — | — | — | 0.5% |
| (Metashine ME 2025 PS | | | | |
| from Nippon | | | | |
| Sheet Glass) | | | | |
| Water qs | 100% | 100% | 100% | 100% |

AM: active material.

Hair treated with these shampoos has good styling and volumizing properties.

1. A composition for washing a keratin material, comprising, in a cosmetically acceptable aqueous medium, at least one insoluble particle with a melting point of greater than 70° C., at least one detergent surfactant chosen from anionic, nonionic and amphoteric surfactants and at least one polymer with a drawing power of greater than 5 cm.

2. The composition according to claim 1, wherein the at least one detergent surfactant is chosen from anionic surfactants.

3. The composition according to claim 1, wherein the at least one detergent surfactant is present in an amount ranging from 4% to 50% by weight relative to the total weight of the composition.

4. The composition according to claim 3, wherein the at least one detergent surfactant is present in an amount ranging from 8% to 25% by weight relative to the total weight of the composition.

5. The composition according to claim 1, wherein the at least one drawing polymer is chosen from:
(a1) a dispersion of particles of at least one water-soluble polymer with a weight-average molecular mass of greater than 106 in a saline aqueous solution, obtained by heterogeneous free-radical polymerization of water-soluble monomers with precipitation of the polymer formed, wherein at least one of the monomers is cationic, and
(a2) an aqueous solution of at least one water-soluble polymer with a weight-average molecular mass of greater than 10⁶, obtained by heterogeneous free-radical polymerization of water-soluble monomers with precipitation of the polymer formed, wherein at least one of the monomers is cationic.

6. The composition according to claim 5, wherein the water-soluble monomers are chosen from at least one of cationic, anionic and nonionic monomers comprising at least one ethylenic double bond, and a mixture thereof.

7. The composition according to claim 6, wherein the anionic monomers are chosen from acrylic acid, methacrylic acid, acrylamido-2-methylpropanesulfonic acid and itaconic acid.

8. The composition according to claim 6, wherein the nonionic monomers are chosen from acrylamide, methacrylamide, N-vinylformamide, N-vinylacetonamide, hydroxypropyl acrylate and hydroxypropyl methacrylate.

9. The composition according to claim 6, wherein the cationic monomers are chosen from di(C₁-₄ alkyl)diallylammonium salts and the compounds of formula (I) 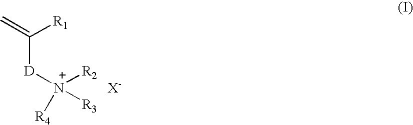
wherein
R₁ is chosen from a hydrogen atom and a methyl group,
R₂ and R₃, which may be identical or different, are each chosen from a hydrogen atom and linear and branched C₁₋₄ alkyl groups,
R₄ is chosen from a hydrogen atom, linear and branched C₁₋₄ alkyl groups, and an aryl group,
D is the following unit 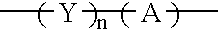
wherein Y is chosen from amide, ester, urethane and urea functional groups,
A is chosen from linear, branched and cyclic C₁₋₁₀ alkylene groups, which may be substituted or interrupted with at least one ring chosen from divalent aromatic and heteroaromatic rings, or which may be interrupted with at least one hetero atom chosen from O, N, S and P, and which may comprise at least one functional group chosen from ketone, amide, ester, urethane and urea functional groups,
n is 0 or 1, and
X⁻ is an anionic counterion.

10. The composition according to claim 5, wherein the at least one water-soluble polymer is polymerized using at least one cationic monomer of formula (I) 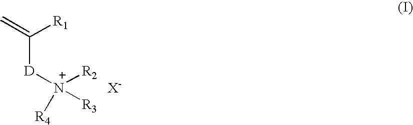
wherein
R₁ is chosen from a hydrogen atom and a methyl group,
R₂ and R₃, which may be identical or different, are each chosen from a hydrogen atom and linear and branched C₁₋₄alkyl groups,
R₄ is chosen from a hydrogen atom, linear and branched C₁₋₄ alkyl groups, and an aryl group,
D is the following unit 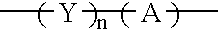
wherein Y is chosen from amide, ester, urethane and urea functional groups,
A is chosen from linear, branched and cyclic C₁₋₁₀ alkylene groups, which may be substituted or interrupted with at least one ring chosen from divalent aromatic and heteroaromatic rings, or which may be interrupted with at least one hetero atom chosen from O, N, S and P, and which may comprise at least one function group chosen from ketone, amide, ester, urethane and urea functional groups,
n is 0 or 1, and
X⁻ is an anionic counterion.

11. The composition according to claim 10, wherein the at least one water-soluble polymer is polymerized using a monomer mixture comprising from 0 to 30 mol % of acrylic acid, from 0 to 95.5 mol % of acrylamide and from 0.5 to 100 mol % of the at least one cationic monomer of formula (I).

12. The composition according to claim 11, wherein the at least one water-soluble polymer is polymerized using a monomer mixture comprising acrylic acid and the at least one cationic monomer of formula (I), wherein the number of moles of the at least one cationic monomer of formula (I) is greater than the number of moles of acrylic acid.

13. The composition according to claim 11, wherein the at least one water-soluble polymer is polymerized using a monomer mixture comprising 10 mol % of acryloyloxyethyldimethylbenzylammonium chloride and 90 mol % of acrylamide.

14. The composition according to claim 11, wherein the at least one water-soluble polymer is polymerized using a monomer mixture comprising 30 mol % of acryloyloxytrimethylammonium chloride, 50 mol % of acryloyloxyethyldimethylbenzylammonium chloride and 20 mol % of acrylamide.

15. The composition according to claim 11, wherein the at least one water-soluble polymer is polymerized using 10 mol % of acryloyloxyethyltrimethylammonium chloride and 90 mol % of acrylamide.

16. The composition according to claim 11, wherein the at least one water-soluble polymer is polymerized using 30 mol % of diallyldimethylammonium chloride and 70 mol % of acrylamide.

17. The composition according to claim 5, wherein the concentration of particles of the at least one water-soluble polymer as a dispersion in a saline aqueous solution in (a1) ranges from 0.01% to 20% by weight, relative to the total weight of the dispersion.

18. The composition according to claim 5, wherein the concentration of the at least one water-soluble polymer as a solution in (a2) ranges from 0.01% to 20% by weight, relative to the total weight of the solution.

19. The composition according to claim 5, wherein at least one of the saline aqueous solutions in (a1) and (a2) comprises at least one anionic salt.

20. The composition according to claim 19, wherein the at least one anionic salt is chosen from ammonium sulfate, ammonium hydrogen sulfate, sodium sulfate, sodium hydrogen sulfate, magnesium sulfate, ammonium sulfate, magnesium hydrogen sulfate, aluminium sulfate and aluminium hydrogen sulfate.

21. The composition according to claim 1, wherein the at least one drawing polymer is present in an amount ranging from 0.01% to 10% by weight relative to the total weight of the composition.

22. The composition according to claim 1, wherein the at least one insoluble particle is chosen from mineral and organic particles.

23. The composition according to claim 1, wherein the at least one insoluble particle is chosen from glass particles, polyamide particles, and optionally expanded polymers and copolymers of acrylonitrile, of vinylidene chloride, of vinyl chloride and of at least one monomer chosen from acrylic and styrene monomers.

24. The composition according to claim 22, wherein the mineral particles comprise an element chosen from columns IIa, IIIa and IVa, IIIb and IVb of the Periodic Table of the elements.

25. The composition according to claim 22, wherein the at least one insoluble particle is chosen from particles comprising at least 10% by weight of calcium carbonate, particles comprising at least 10% by weight of at least one silicate, particles comprising at least 90% of at least one compound chosen from aluminium oxide, silicas, magnesium oxide, zinc oxides, titanium oxides and barium sulfate, and mixtures thereof.

26. The composition according to claim 1, wherein the at least one insoluble particle is present in an amount ranging from 0.001% to 20% by weight relative to the total weight of the composition.

27. The composition according to claim 1, further comprising at least one cationic polymer other than the at least one drawing polymer.

28. The composition according to claim 27, wherein the at least one cationic polymer is present in an amount ranging from 0.001% to 20% by weight relative to the total weight of the composition.

29. The composition according to claim 1, further comprising at least one silicone.

30. The composition according to claim 29, wherein the at least one silicone is present in an amount ranging from 0.01% to 20% by weight relative to the total weight of the composition.

31. The composition according to claim 1, wherein the composition has a pH ranging from 3 to 10.

32. A method for treating hair, comprising applying to the hair a composition comprising, in a cosmetically acceptable aqueous medium, at least one insoluble particle with a melting point of greater than 70° C., at least one detergent surfactant chosen from anionic, nonionic and amphoteric surfactants and at least one polymer with a drawing power of greater than 5 cm, wherein the treatment is chosen from cleaning, caring for, conditioning, and styling the hair.
